(19) United States
(12) Patent Application Publication
(10) Pub. No.: US 2021/0043302 A1
(43) Pub. Date: Feb. 11, 2021

(54) HEALTH MANAGEMENT SYSTEMS AND METHODS

(71) Applicant: Zhendong Song, Rochester, MN (US)

(72) Inventor: Zhendong Song, Rochester, MN (US)

(73) Assignee: mSense, Rochester, MN (US)

(21) Appl. No.: 16/695,716

(22) Filed: Nov. 26, 2019

Related U.S. Application Data
(60) Provisional application No. 62/884,542, filed on Aug. 8, 2019.

(51) Int. Cl. G16H 20/30; G16H 40/67; G16H 20/10

(52) U.S. Cl. CPC G16H 20/30; G16H 20/10; G16H 40/67

(57) ABSTRACT

A health management system is disclosed. The health management system can include a smart health tracking device, a managing console device in communication with the smart health tracking device, and a voice assistant in communication with the managing console device. The smart health tracking device can also measure and store vital statistical data regarding the health condition of a user. Data collected from the smart health tracking device is stored in an internet database.

### RELATED APPLICATIONS

This application claims benefit to U.S. Provisional Patent Application Ser. No. 62/884,542, filed Aug. 8, 2019, and titled, MEDICATION MANAGEMENT, which is herein incorporated by reference in its entirety.

### FIELD

This disclosure relates to intelligent medication and health device management. This disclosure further relates to a system and method of tracking adherence to medication schedules and scheduled health testing in real-time.

### BACKGROUND

A large number of people (patients) take medications and/or collect health data for various health conditions. With respect to medications, medications are most effective when taken in the dosage and at the intervals suggested by health personnel such as physicians, dentists, physician assistants, nurses, pharmacists, or other health professionals. In some circumstances, taking the wrong medication, the wrong dosage of the right medication, or taking medication at the wrong time can make the medication less effective, ineffective, or even can cause further health problems of a patient. In the case of health regimens, some patients have the need to monitor data such as, for example, blood pressure, weight, glucose levels, and other health data that can be collected, for example at home. This health data can be important to a health professional in order to administer a treatment plan that can include a medication regimen. In addition, nonadherence to health regimens, such as those described above, costs the health system around $300 billion each year and can result in worse outcomes than adherence to a health regime. Sometimes, nonadherence to the health regimen can result in the need for more physician visits, possible hospitalization, readmission to the hospital, or a worsening in the condition being treated.

Much nonadherence to health or medication regimens is avoidable. It is common for patients to forget to take medication or to not remember if they have taken medication. This can result in, on the one hand, not taking enough medication for the prescribed ailment or, on the other hand, double dosing on a medication. Both of these scenarios may be dangerous to the patient. Sometimes, caregivers who provide for patients can be unaware that the patient has run out of medication and needs it refilled.

### SUMMARY

There is a need for health management systems that overcome some of the shortcomings listed above. There is a need for methods and systems to track the adherence of a patient to a medication regimen whether prescribed or not. Additionally, there is a need for systems that can track vital statistics that need to be collected for a patient at regular time intervals. There is also a need for methods and systems that allow for the monitoring of patients which can help patients stay on proper medication or health tracking regimens by informing caregivers, if present, health professionals, or patients themselves, that either they are adhering to the regimen or that they have deviated from that regimen and need a reminder, refill, consultation with a health professional, help with a medication, or any of a number of other issues surrounding medication management.

In one aspect, a health management system is disclosed that includes a smart health tracking device, a managing console device in communication with the health tracking device, and a voice assistant in communication with the managing console device or the smart health tracking device. In some embodiments, at least one of the smart health tracking device, the managing console device, or the voice assistant includes a wireless connection. In some other embodiments, the disclosed health management system includes an internet database that may be a private or public internet database. The disclosed health management system can track a user's adherence to a medical regimen. The disclosed health management system can also include an accelerometer.

In another aspect, a method of health management is disclosed that can includes connecting a user through a voice assistant to one or more smart electronic computing devices. The disclosed health management method can further include connecting the one or more smart electronic computing devices to an internet database. Information regarding health management of the user can be stored in the internet database. The method also can includes analyzing the information in the internet database and, optionally, having the information interact with a provider. Additionally, the disclosed method also can include outputting the analyzed information to a voice assistant wherein the voice assistant provides specific instructions to the user.

In yet another aspect, a health management system is disclosed that can include more than one internet-connected health device connected to a computer network. An internet database can be connected to the computer network. The disclosed health management system can also include a smart communication device connected to the internet database. The internet database can receive and store data from at least one of the more than one internet-connected health devices, and wherein the smart communication device can send health data about a patient to the patient or a health professional. In some embodiments the internet-connected health device can include a smart pillbox, a smart pill bottle, a smart blood pressure monitor, a smart glucose monitor, or a combination thereof. Additionally, the internet-connected health device can include a device that measures health data of a patient. In some embodiments, the disclosed health management system can send out an alert.

The disclosed health management system and method can help a user avoid nonadherence to a health or medication regimen recommended by a medical professional. In addition, the disclosed health management system can use sensor and internet technology to help track medication and make sure that medication is taken at the proper time in the proper amount. The disclosed health management system can visually and audibly interact with a user, a provider, a caregiver, or a medical professional.

In this application the term:

“health data” refers to data that can be collected by a user such as, for example, blood pressure, weight, and glucose levels;

“intelligent” refers to devices or systems that can interact with each other and share information between them and in interaction with an internet database or cloud database;

“health condition” refers to a syndrome of symptoms that a patient or user may display;

“health professional” refers to a person that recommends a health regimen to a patient and can include, but is not limited to, a physician, physician's assistant, nurse, dentist, physical therapist, or any other person recommending a treatment for a health condition;

“health regimen” refers to a process provided by a health professional to be followed by a patient and includes, for example, taking medication, performing physical therapy, measuring vital statistics, interacting with the health professional, visiting the health professional, or performing hygiene procedures that need to be done on a scheduled basis;

“medication” refers to physical medicines, such as pills, injections, ointments, or other medicaments and also refers to health regimes;

“medication adherence” refers to health behavior data and deals with how a patient follows health protocols provided by a medical professional;

“smart electronic devices” refer to interactive electronic gadgets that can understand simple commands sent by users and can include, for example, smartphones, tablets, phablets, smartwatches, smart glasses, and can include electronic virtual assistants;

“user” is used interchangeably with “patient”, “provider”, or “caregiver” in that in some embodiments, the user may be a patient, provider, or caregiver; and

“voice assistant”, also known as an “intelligent virtual assistant” or an “intelligent personal assistant” refers to a software agent that can perform tasks or services based upon voice commands or questions;

Features and advantages of the present disclosure will be more readily understood from the following detailed description which should be read in conjunction with the accompanying drawings.

### BRIEF DESCRIPTION OF THE DRAWINGS

The drawings are schematic illustrations and are not intended to limit the scope of the invention in any way. The drawings are not necessarily to scale.

FIG. 1 is a schematic diagram of an embodiment of the health management system disclosed herein.

FIG. 2 is a schematic flow diagram of an embodiment of a portion of the health management system disclosed herein.

FIG. 3 is a schematic flow diagram of an embodiment of a portion of the health management system disclosed herein.

FIG. 4 is a schematic flow diagram of an embodiment of a portion of the health management system disclosed herein.

FIG. 5 is a schematic flow diagram of an embodiment of a portion of the health management system disclosed herein.

FIG. 6 is a schematic flow diagram of an embodiment of a portion of the health management system disclosed herein.

### DETAILED DESCRIPTION

In the following detailed description, embodiments will be described in detail with reference to the drawings, wherein like reference numerals represent like parts and assemblies throughout the several views. Reference to various embodiments does not limit the scope of the claims attached hereto. Additionally, any examples set forth in this specification are not intended to be limiting and merely set forth some of the many possible embodiments for the appended claims. It is to be understood that various omissions and substitutions of equivalents are contemplated as circumstances may suggest or render expedient, but these are intended to cover applications or embodiments without departing from the spirit or scope of the claims attached hereto. Also, it is to be understood that the phraseology and terminology used herein are for the purpose of description and should not be regarded as limiting.

Since following a health regimen can be an important and frequent event, what is needed is a health management system that can address current shortcomings such as those mentioned above. Some of the causes of nonadherence to a health regimen are listed above and include, for example, forgetting to take a proper medication at the proper time and in a proper dose, double dosing from taking more than the proper dose, running out of medication, forgetting to keep required appointments with a health professional, and failing to do prescribed physical therapy routines. In some cases, nonadherence to health regimens can also include, for example, oral health management, mental health program attendance, chemical dependency treatment management, or other suggested or recommended treatment programs.

Using sensor and internet technology, adherence to a health regimen can be tracked in time and monitored by a patient, a health professional, or a caregiver. In some embodiments, the tracking can be done in real-time. Medication tracking devices such as internet-connected smart pill boxes, pill bottles, or bottle caps are commercially available. In addition, smart vital statistic measuring devices such as smart blood pressure monitors, smart scales, and smart glucose monitors are available that can measure vital statistics of a user. The smart devices can detect and transmit information regarding when a medication has been taken, but don't provide a convincing user interface. They can operate in conjunction with various commercial wireless systems such as BLUETOOTH, ZIGBEE, LORA, WI-FI, or cellular phone service. Most commercial devices detect the opening and closing of pill boxes—some use a weight measuring device, such as a scale, to measure the exact amount of medication to be dispensed at each dosing time. Many of the available systems can use audio or visual cues on the tracking devices to remind patients to take medication, to alert the risk of double dosing, to initiate automatic call/text messages to caregivers or health professionals and/or transmit data to health professionals when nonadherence is detected. Most of these available systems provide an app to help set up the tracking device and can display medication adherence history.

A health management system with a smart medication tracking device and a voice assistant, as two separate devices, has not been disclosed nor has a medication management system been disclosed where a medication tracking device and a voice assistant are located remote from each other—across the room, in another room, in another building, or even in another geographical location. Additionally, a health management system with a smart vital statistic tracking device and a voice assistant, as two separate devices, has not been disclosed nor has a health management system been disclosed where a smart medication tracking device or a smart vital statistic measuring device and a voice assistant are located remote from each other across the room, in another room, in another building, or even in another geographical location. Additionally, a process as to how these two (or more) devices interact has not been disclosed.

In one aspect, a health management system is disclosed. In some embodiments, the health management system can include a smart health tracking device, a managing console device in communication with the smart health tracking device, and a voice assistant in communication with the managing console device. The smart health tracking device, in some embodiments, can measure and store vital statistical health data of a user. In some embodiments, the smart health tracking device can be a smart medication tracking device that can dispense medications. The smart medication tracking device can, for example, dispense a single dose of medication at an appropriate, scheduled time as directed by the managing console device. The smart medication tracking device can then communicate with the managing console device or an internet database regarding information relating to the time and dosage of medication that it has dispensed, whether the medication needs refilling. The smart medication tracking device can also detect if the container that contains the medication has been opened or closed and if the weight of the medication has changed. The smart medication tracking device can store and transmit a dosing record of the type and amount medication that is dispensed and can transmit the information to the managing console device. The smart health tracking device can also include a wireless connection. In some embodiments, the smart health tracking device can be connected to the internet (via, for example, a wireless connection) or a local private network. In these embodiments, the smart health tracking device can interact with users, regardless of location, to provide an optimal and customized experience for the user.

The managing console device can be a smart electronic device in communication with the health tracking device. In some embodiments, the managing console device can be a smartphone. The graphic user interface (UI) in smartphones make them easy to set up and view dosing history. However, a smartphone may not be in proximity to the user to effectively alert/remind the user of information relating to health regimen or medication management. In some embodiments, the smart electronic device can be a smartphone, smart tablet, or a portable electronic computing device. In some embodiments, the smart electronic device can be device dedicated to the health management system. The managing console device can communicate with the smart medical tracking device. The communication can include setting up a medication adherence schedule and a dosing history of the medication. The managing console device may also relay information between the tracking device or devices and a database, for example, through a WiFi, cellular, or BLUETOOTH-connected pillbox. The database can be kept locally on the smart electronic device, on a web-based application located remotely, and/or on a cloud database. For example, the managing console device can uplink dosing records from the managing console device to a smartphone and then to a database, such as a cloud database. Alternatively, the managing console device can update firmware remotely as initiated from the cloud, send installation information to the smartphone and then upgrade the devices. The managing console device can also be used to set up a voice assistant, such as a smart speaker.

The cloud information database can store device setup parameters, schedule time for medication, dosing history, and record any interactions between users (for example, patients, or other authorized persons, health professionals), a smartphone and, but not limited to, the managing console device. Typically, the cloud receives information from the managing console or the smart medical tracking device. The cloud can calculate the amount of medication acquired by the user and the amount of remaining medication based upon actual dosing history. Additionally, the cloud database can suggest prescription refill and contact the user, a health professional, or a pharmacist directly. The cloud information database can also store vital statistical data collected from smart health devices that can measure the health data of users at regular intervals.

In some embodiments, information stored or retrieved from the internet database can include a user's dosing record, a medication schedule, a scheduled time for medication, information from a provider, a record of interaction between the user and the medication tracking device, an amount of medication in the medication dispensing device, or a combination thereof. It is also contemplated that any information in audio/visual format can be transmitted from the provider to the user to enhance the user's (or the caretaker's) knowledge. Such information can include, for example, educational materials in the form of a video.

The disclosed health management system also includes a voice assistant. In some embodiments, the voice assistant can be a smart speaker such as, for example, ALEXA (Amazon's cloud-based voice service) or GOOGLE ASSISTANT (Google's cloud-based service), or many others that are commercially available. The voice assistant can communicate with the patient in an interactive manner. The voice assistant can also access the database and can be programmed to wake up, interact with the patient and record necessary information from the interaction. It can send this information to the database. In some embodiments, the voice assistant can be programmed via the internet database or a cloud database and/or the managing console device to interact with a user. For example, the user can ask the voice assistant if he/she has taken the last dose of medication. The voice assistant can access the database and inform the user the last time that the medication was taken. In some embodiments, multiple medications, in separate containers, can be stored in the same dispensing system (i.e., smart pillbox or smart pill bottle) and the voice assistant can announce which medication container has been opened. When it is time to take the next dose of medication there is no need to remind the user again. But if the user has not taken the medication at that time, the voice assistant can alert the user and use a voice to issue a reminder to the user that it is time to take the medication. This reminder can be made in combination with an audio or a visual alert from the medication tracking device.

In some embodiments, a smart speaker can be set up to access information such as, for example, a medication name (generic name or trademark name), dosing schedule, dosing history, or any additional information provided by a health professional. Voice assistants, such as smart speakers, have become very popular. Voice-based user interfacing (UI) is easy and convenient to use and has the potential to provide excellent interactions with users by providing more engaging experiences than monotonic buzzing sounds, flashing LED lights, robot-calls or text messages. Most importantly, better patient engagement with a medication management system can result in effective adherence to medication regimens.

If the user has taken a proper dose of medication but has forgotten about taking it, the device can alert the user (or a caretaker). An alert system of this type can be useful, for example, for patients that have memory loss, such as those suffering from early dementia. The alert can be issued, for example, when a medication dispenser has been opened. The event of a user opening the medication dispenser can be transmitted to the database in real time and it may trigger preset criteria setting a threshold for undesirable occurrences such as double dosing (for example, a medication container being opened for a second time within 30 minutes or any other preset criteria). The voice assistant can ask the user (patient) if he/she really wants to medication since it has been alerted that the medication dispenser has been opened within the last preset period of time.

In some other embodiments, an accelerometer can be built into the medication tracking device to detect and track motion. For example, when the user picks up or opens the medication container in the medication tracking device, that information can be sent to the cloud database to check for double dosing. If the medication was taken shortly before the user picks up or opens the medication container in the medication tracking device, the cloud database can trigger the voice assistant to notify the user or a caregiver of the potential risk of double dosing.

In some embodiments, a health management system is disclosed that interacts with an internet database and retrieves information sent to it from the smart health tracking device, the smart electronic device, the voice assistant, the medication dispenser, an outside provider, the managing console device, or a combination thereof. In some embodiments, the managing console, the smart health tracking device, the voice assistant, or any combination thereof can be located remote from each other. For example, in some embodiments, the patient may be in voice proximity to the voice assistant, but the smart health tracking device or the managing console device can be in another room, another building, or even another geographical location (for example, in another community, city, state, or even country). In another example, a caretaker can purchase a smart medication dispenser for a patient located in a different location as the caretaker. The caretaker can set up the medication dispenser in proximity to the patient with a voice assistant located in proximity of the caretaker. Then the caretaker can use the voice assistant to query the patient if the patient has taken the proper medication at the proper time. The voice assistant can then remind the caretaker if the medication has been taken improperly. Furthermore, the caretaker can initiate a call to the patient using the voice assistant.

Some additional features of the disclosed health management system are as follows. If desired, the voice user interface of the voice assistant can be modified locally or remotely. The dosing history, adherence history, and/or behavior patterns of the users can be summarized by the system and, with permission, can be delivered to the users or other authorized personnel such as providers or caregivers. In some embodiments, content information suggested by a health professional, an informational website, or a provider can be verbally read to the patient, or, if a video screen is available either on or connected to the managing console device, can be displayed on the video screen. In some other embodiments, it is contemplated that content information can be printed out from the health management system if a printer is attached. In some other embodiments, authorized personnel (such as, for example, a health professional) can leave a message for the user remotely. For example, via a smartphone application, or by logging into a website. Additionally, the message can be played from a voice assistant connected to the medication management system. In some embodiments, information stored in the internet database can include amount of medication (i.e., number of pills) remaining and when it is time (with appropriate leeway) to order a prescription refill or contact the health professional to reauthorize a prescription refill. In some embodiments, a message can be provided and played to a user via a smartphone and/or smart speaker. In some embodiments, a user can order the refill through the smart speaker without having to contact the providing pharmacy directly. In some embodiments, the internet database (i.e., cloud database) can be programmed so that if the medication tracking device detects that the medication container has been opened at a time other than the normal, scheduled time for taking a medication, a message can be sent automatically to the user's managing console device that can be, for example, a smartphone, and/or a voice assistant.

In another aspect, a health management system is contemplated in which multiple internet-connected devices such as a smart pillbox, a smart pill bottle, a smart blood pressure monitor, a smart glucose monitor, or other smart health devices manage the many aspects of a patient's health. A smart device is capable of communicating in one form or another with a user (that includes, for example, a patient, provider, or caregiver) and with a network—local or internet-based. In embodiments of this aspect, each smart device of multiple devices can connect directly to the internet through, for example, a wireless hub. In some embodiments, individual BLUETOOTH-enabled monitors can be connected to a wireless hub with both BLUETOOTH and WiFi capability. Vital statistics collected from each of the multiple smart devices can be uploaded to an internet database, such as a cloud database. Alternatively, each of the smart monitoring devices can function as a wireless hub, itself. For example, a smart pillbox can connect with compatible monitoring devices such as a BLUETOOTH-enabled blood pressure monitor, a BLUETOOTH-enabled scale. Vital statistics, for example, recorded blood pressure, data from a glucose monitor, and/or weight data along with any medication data in the system can be routed and uploaded to the internet database using the smart pillbox (through its WIFI connection). In some embodiments, the smart pillbox can have a locking device that can be remotely controlled by the health management system.

In some embodiments, any alerting information stored in the internet database, an alert that blood pressure is trending high or that a scheduled blood pressure has not been measured for a long time period or does not conform to a provider's recommendation, can be sent to the user, a provider, or a caretaker (alternatively a health professional). Based upon this alerting information, a smart voice assistant can instruct the user or patient to perform certain tasks such as taking a blood pressure measurement, inquire if the user is feeling OK, or tell the user that recent health adherence has not been in accordance with the provider's recommendations or that blood pressure has been trending high. The medication and health managing system can then communicate further with the user and health professionals to offer remedies to the alerted condition or conditions.

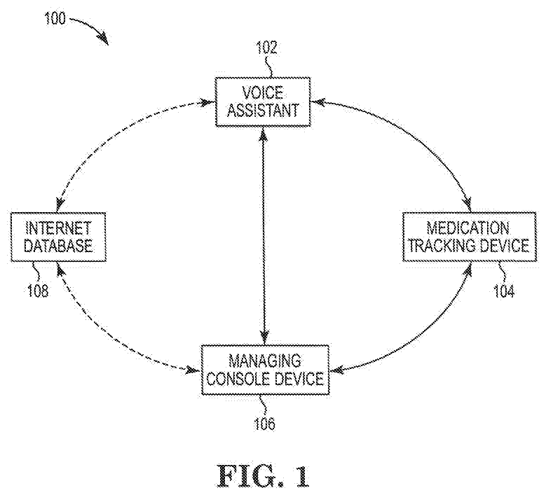

The medication and health management system and method disclosed herein can be better understood by the enclosed drawings. Turning to FIG. 1, health management system 100 includes health tracking device 104 that is in communication with managing console device 106 and voice assistant 102. Consequently, managing console device 106 is also in communication with voice assistant 102. In some embodiments, voice assistant 102 and medication tracking device 104 are separate devices as shown in FIG. 1. In some embodiments, smart medication tracking device 104 includes a wireless connection that allows it to interact with internet database 108. Internet database 108 can be a local database, a network database such as a LAN (local area network), a WAN (a wide area network), or a cloud database. In some embodiments, voice assistant 102 can be a smart speaker as described above. Voice assistant 102 can also include a wireless connection allowing it to communicate with other components of the system including, for example, internet database 108.

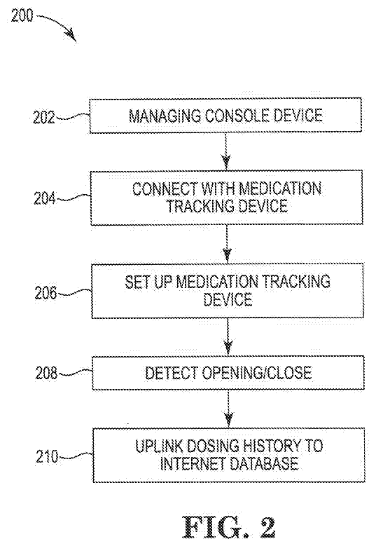

In an embodiment 200 illustrated in FIG. 2, managing console device 202 connects with the smart medication tracking device directly, as shown in 204. This connection can be made, for example, through a BLUETOOTH connection or via a WIFI connection to the internet, accessing the internet database. When connected to the smart medication tracking device, managing console device 202 can set up the medication tracking device 206 and can, for example, set up a connection with the other components (as shown in FIG. 1), determine the time for a patient taking medication by querying an internet database, determine a weekly schedule for taking medication, set up alerts, mute itself, or any other useful functions. In an important specific embodiment, managing console device 202 can directly the activities of a smart pillbox such as, for example, by detecting the opening or closing of medication available from a medication tracking device as shown in 208. Finally, the medication management system can upload dosing history of medication to the internet database as shown in 210.

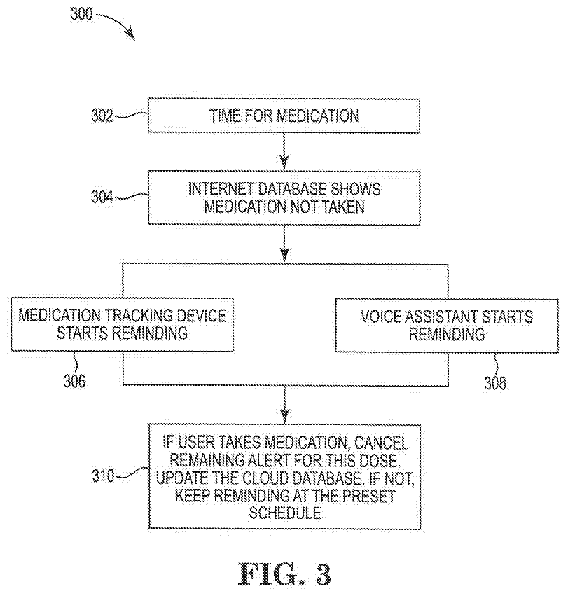

FIG. 3 is a schematic flow diagram that shows what can happen when it is time for a patient to take medication. Flow diagram 300 shows the disclosed medication management system monitoring time for medication 302 for a patient. In the illustrated embodiment, the internet database indicates that a patient has not taken the proper mediation at the proper time, in the proper amount, or as has been recommended by the health professional 304. The disclosed medication management system then can use a medication tracking device to start reminding the patient to take the proper mediation at the proper time, in the proper amount, or as has been recommended by the health professional as shown in 306. Alternatively, or concomitantly, the voice assistant can also begin reminding the patient to take the medication as shown in 308. As shown in 310, if the user or patient takes the medication, the disclosed medication management system can cancel any future alerts and update the internet database. If the medication has not been taken the system can keep reminding the patient of user at preset schedule times.

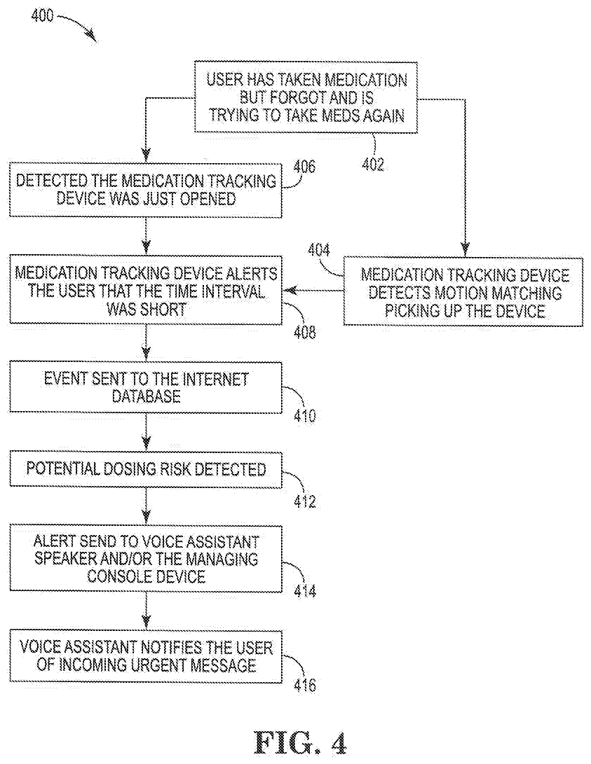

FIG. 4 is a schematic flow diagram of another scenario 400 similar to that shown in FIG. 3 but where the patient (or user) has forgotten that they took medication and are trying to take the medication again (double dose) 402. This scenario can occur when the medication tracking device has detected that a device containing medication has been opened 406. Alternatively, as shown in 404, the medication tracking device can detect motion and that the motion matches that expected when a patient picks up a device such as a medication container. This condition can occur, for example, by the use of an accelerometer. The medication tracking device can alert the user 408 that the time interval was too short to take the medication again. If this condition occurs, this information can be sent to the internet database 410 and it can be determined if a potential double dosing risk has been detected 412. If such a determination has been made, an alert can be sent to the voice assistant and/or the managing console device 414. Then the voice assistant, for example, can alert the user of an upcoming urgent message 416 with the double dosing alert and the remedy for the double dosing alert.

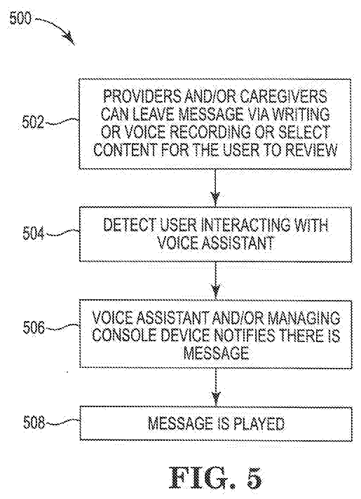

FIG. 5 is a schematic flow diagram of an embodiment of a portion of the health management system disclosed herein. Flow diagram 500 shows another embodied feature of the provided medication management system. Providers and/or caregivers can leave a message in writing or by voice recording 502 in response to either an alert as shown in FIG. 4 or at the request of a patient, user, or caregiver. In some embodiments, the provider and/or caregivers can select relevant content. The system can detect a user interacting with the voice assistant as shown in 504. The voice assistant and/or the managing console device can notify the patient, user, or caregiver if there is a message posted 506. Finally, the message can be played 508.

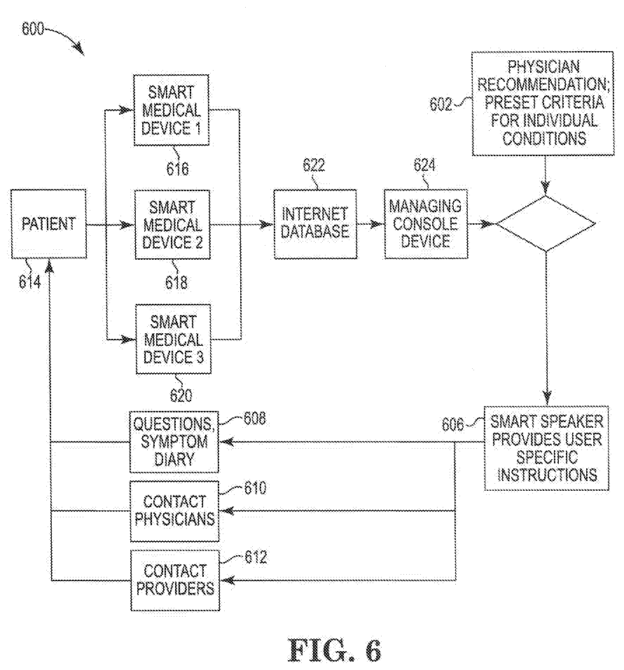

FIG. 6 is a schematic diagram of an embodiment of a portion of the health management system disclosed herein. Health management system 600 includes, in the illustrated embodiment, three smart health devices: 616 (smart health device 1), 618 (smart health device 2), and 620 (smart health device 3). In some embodiments, smart health device 616 can be a smart blood pressure monitor, smart health device 618 can be a smart scale, and smart health device 620 can be a smart glucose monitor. In the embodiment illustrated in FIG. 6, smart health devices 616, 618, and 620 are interconnected and they also are connected to internet database 622. In other embodiments, not all of the smart health devices in a system such as that shown in FIG. 6 need to be interconnected. In some embodiments the interconnections and connections can be wireless and smart health devices 616, 618, and 620 as well as internet database 622 may be located in different places.

Smart health devices 618, 620, and 622 measure or collect data and interact with patient 614. Information stored in internet database 622 (for example, medication adherence data or vital statistical data) can be collected (fused) and analyzed by managing console device 624. This information can then be reviewed by health professional 602 who can preset criteria depending upon data that has been collected. The information from health professional 602 and managing console device 624 can then be relayed to voice assistant 606. Voice assistant 606 can be located remotely from any of the components shown in FIG. 6. Voice assistant 606 can provide user specific instructions for completing certain tasks such as taking a medication regimen or keeping track of certain vital statistics at a prescribed interval. Finally, upon interaction with voice assistant 606, health management system 600 can answer questions about symptoms or other health concerns 608, contact health professionals for more follow-up information 610, or contact providers or caregivers, or authorized personnel 612 for interaction with patient 614.

While embodiments of the invention have been illustrated and described, it will also be apparent that various modifications can be made without departing from the scope of the invention. It is also contemplated that various combinations or sub combinations of the specific features and aspects of the disclosed embodiments can be combined with or substituted for one another in order to form varying modes of the invention. Accordingly, it is not intended that the invention be limited, except as by the appended claims. All references cited within are herein incorporated by reference in their entirety.

What is claimed is:

1. A health management system comprising:
a smart health tracking device;
a managing console device in communication with the health tracking device; and
a voice assistant in communication with the managing console device or the health tracking device.

2. A health management system according to claim 1, wherein at least one of the smart health tracking device, the managing console device, or the voice assistant comprises a wireless connection.

3. A health management system according to claim 1, wherein the voice assistant and the smart health tracking device are separate devices.

4. A health management system according to claim 2, further comprising an internet database in communication with the health tracking device through the wireless connection.

5. A health management system according to claim 4, wherein the internet database stores and retrieves information sent to it from the medical tracking device, the smart electronic device, the voice assistant, the medication dispenser, an outside provider, the managing console device, or a combination thereof.

6. A health management system according to claim 5, wherein the information comprises a user's dosing record, a medication schedule, a scheduled time for medication, information from a provider, a record of interaction between the user and the smart health tracking device, an amount of medication in the medication dispenser, or a combination thereof.

7. A health management system according to claim 6, wherein the information comprises a record of a user's adherence to a medical regimen.

8. A health management system according to claim 7, wherein the user interacts with the voice assistant to determine the last time medication was dispensed to the user or the last time the user completed the medical regimen.

9. A health management system according to claim 8, wherein system alerts the user if the medication has not been taken at the proper time and at the proper dose or the medical regime has not been completed.

10. A health management system according to claim 1, further comprising an accelerometer.

11. A health management system according to claim 2, wherein the managing console device supplies information relating to medication management to the user or another authorized person.

12. A health management system according to claim 11, wherein educational materials can be provided to a user from the medication management system.

13. A health management system according to claim 12, wherein the educational materials comprise a video.

14. A health management system according to claim 2, wherein the smart health tracking device tracks health data.

15. A method of health management comprising:
connecting a user through a voice assistant to a one or more smart electronic computing devices;
connecting the one or more smart electronic computing devices to an internet database;
storing information regarding health management of the user in the internet database;
analyzing the information in the internet database;
optionally, having the information interact with a provider;
outputting the analyzed information to a voice assistant wherein the voice assistant provides specific instructions to the user.

16. A health management system comprising:
more than one internet-connected health device connected to a computer network;
an internet database connected to the computer network; and
a smart communication device connected to the internet database,
wherein the internet database receives and stores data from at least one of the more than one internet-connected health devices, and
wherein the smart communication device sends health data of a patient to the patient or a health professional.

17. A health management system according to claim 16, wherein the internet-connected health device comprises a smart pillbox, a smart pill bottle, a smart blood pressure monitor, a smart glucose monitor, or a combination thereof.

18. A health management system according to claim 16, wherein the internet-connected health device measures health data of a patient.

19. A health management system according to claim 16, wherein the system sends out an alert.
